(19) United States
(12) Patent Application Publication
(10) Pub. No.: US 2026/0033790 A1
(43) Pub. Date: Feb. 5, 2026

(54) COMPUTED TOMOGRAPHY DEVICE WITH A RADIATION PROTECTION APPARATUS FOR COVERING AN OPENING

(71) Applicant: Siemens Healthineers AG, Forchheim (DE)

(72) Inventors: Martin RAMSAUER, Pyrbaum (DE); Benjamin LOEWEN, Schwaebisch Gmuend (DE); David DEDERICHS, Poxdorf (DE); Jens FEHRE, Hausen (DE); Daniel KRIEGER, Pommersfelden (DE)

(73) Assignee: Siemens Healthineers AG, Forchheim (DE)

(21) Appl. No.: 19/273,317

(22) Filed: Jul. 18, 2025

(30) Foreign Application Priority Data
Aug. 2, 2024 (EP) 24192603.9

(51) Int. Cl. A61B 6/10; A61B 6/03; A61B 6/04

(52) U.S. Cl. CPC A61B 6/107; A61B 6/03; A61B 6/0407

(57) ABSTRACT

A computed tomography device comprises a gantry with an opening, and a radiation protection apparatus covering the opening. The radiation protection apparatus has a holding structure, a radiation protection curtain and a pivoting apparatus. An examination object can be inserted into the opening along a system axis of the gantry when the holding structure is in a first position relative to the gantry. The holding structure is pivotably mounted relative to the gantry about a pivot axis via the pivoting apparatus such that the holding structure can be lowered relative to the gantry from the first position to a second position by a first pivoting motion of the holding structure about the pivot axis. The radiation protection curtain is arranged on the holding structure. A lower region of the radiation protection curtain hangs down from the holding structure.

### CROSS-REFERENCE TO RELATED APPLICATION(S)

The present application claims priority under 35 U.S.C. § 119 to European Patent Application No. 24192603.9, filed Aug. 2, 2024, the entire contents of which is incorporated herein by reference.

### FIELD

One or more example embodiments of the present invention relate to a computed tomography device (CT device).

### BACKGROUND

Mobile computed tomography devices are increasingly being used in environments that were not originally designed for operating a device that emits ionizing scatter radiation. Examples of such environments include intensive care units, operating and intervention rooms and mobile stroke units. To protect the environment of the computed tomography device from scatter radiation, the paneling of the computed tomography device can have layers of a radiation protection material, for example lead. Measures are also required to protect the environment of the computed tomography device from scatter radiation emitted from an opening of the computed tomography device, for example at a front side or rear side of a gantry of the computed tomography device. It should be possible to adapt these radiation protection measures as flexibly as possible to the space requirement for body parts and/or accessories connected to the examination object.

US 8 057 097 B1, US 2021/0 369 214 A1 and US 2023/0 094 920 A1 should be mentioned here as prior art.

### SUMMARY

An object of one or more example embodiments of the present invention is to enable improved protection for the environment of a computed tomography device against scatter radiation emitted from an opening of a gantry of the computed tomography device. All subject matter of the independent claims achieves this object. Further advantageous aspects of example embodiments of the present invention are considered in the dependent claims.

Independent of the grammatical term usage, individuals with male, female or other gender identities are included within the term.

One or more example embodiments of the present invention relate to a computed tomography device having a gantry with an opening and a radiation protection apparatus for covering the opening,
wherein the radiation protection apparatus has a holding structure, a radiation protection curtain and a pivoting apparatus,
wherein an examination object can be inserted into the opening along a system axis of the gantry when the holding structure is in a first position relative to the gantry,
wherein the holding structure is pivotably mounted relative to the gantry about a pivot axis via the pivoting apparatus in such a way that the holding structure can be lowered relative to the gantry from the first position to a second position by a first pivoting motion of the holding structure about the pivot axis,
wherein the radiation protection curtain is arranged on the holding structure, in particular attached to the holding structure,
wherein a lower region of the radiation protection curtain hangs down from the holding structure.

One embodiment provides that an upper region of the radiation protection curtain rests on the holding structure.

One embodiment provides that the upper region of the radiation protection curtain rests on the holding structure in such a way that the upper region of the radiation protection curtain shields a region of an environment of the gantry from scatter radiation from X-rays which, emanating from the opening, are directed toward the region of the environment of the gantry and penetrate the holding structure, in particular when the holding structure is in the second position of the holding structure.

One embodiment provides that the lower region of the radiation protection curtain and the upper region of the radiation protection curtain adjoin one another along an edge of the radiation protection curtain, wherein the radiation protection curtain is arranged on the holding structure in such a way that the edge of the radiation protection curtain extends along a first edge of the holding structure.

In particular, it can be provided that a vertical position of the first edge of the holding structure is lower in the second position of the holding structure than in the first position of the holding structure. In particular, the first edge of the holding structure can be located on a side of the holding structure facing away from the pivot axis. The pivot axis can, for example, be located in a region of a second edge of the holding structure. In particular, it can be provided that the upper region of the radiation protection curtain extends from the first edge of the holding structure to the pivot axis and/or to the second edge of the holding structure.

One embodiment provides that the lower region of the radiation protection curtain and the upper region of the radiation protection curtain are sewn together at the edge of the radiation protection curtain. The radiation protection curtain can, for example, be produced by sewing a piece forming the lower region of the radiation protection curtain and a piece forming the upper region of the radiation protection curtain together at a joint forming the edge of the radiation protection curtain.

One embodiment provides that the holding structure extends flat, wherein the upper region of the radiation protection curtain rests flat on the holding structure. One embodiment provides that the holding structure is made of plastic, in particular carbon fiber-reinforced plastic. This allows the holding structure to be lightweight, stable and easy to clean. The holding structure can, for example, be embodied in the form of a lid, in particular in the form of a fully closed and/or upwardly curved lid.

One embodiment provides that the radiation protection curtain is attached to the holding structure via a set of pushbutton connections. This allows the radiation protection curtain to be easily and securely attached to and/or detached from the holding structure.

One embodiment provides that the radiation protection curtain has a set of curtain-side connecting elements, wherein the holding structure has a set of holding-structure-side connecting elements, wherein the set of curtain-side connecting elements and the set of holding-structure-side connecting elements together form the set of pushbutton connections.

In particular, it can be provided that the upper region of the radiation protection curtain has the set of curtain-side connecting elements and/or that an upper side of the holding structure has the set of holding-structure-side connecting elements. In particular, it can be provided that the curtain-side connecting elements are pushbutton heads and/or that the holding-structure-side connecting elements are pushbutton recesses for receiving the pushbutton heads. Other arrangements of pushbutton recesses and pushbutton heads are also possible in principle.

One embodiment provides that the set of holding-structure-side connecting elements has a first row of holding-structure-side connecting elements, wherein the first row of holding-structure-side connecting elements is arranged following a course of the first edge of the holding structure, wherein the set of curtain-side connecting elements has a first row of curtain-side connecting elements, wherein the first row of curtain-side connecting elements is arranged following a course of the edge of the radiation protection curtain and corresponding to the first row of holding-structure-side connecting elements.

In particular, it can be provided that the set of holding-structure-side connecting elements has a second row of holding-structure-side connecting elements, wherein the second row of holding-structure-side connecting elements is arranged following a course of the second edge of the holding structure, wherein the set of curtain-side connecting elements has a second row of curtain-side connecting elements, wherein the second row of curtain-side connecting elements is arranged following a course of the edge of the radiation protection curtain and corresponding to the second row of holding-structure-side connecting elements.

One embodiment provides that the pivoting apparatus has at least one friction hinge, wherein the holding structure is pivotably mounted relative to the gantry about the pivot axis via the at least one friction hinge, wherein the at least one friction hinge is configured to hold the holding structure in the second position relative to the gantry, in particular by a breakaway torque of the friction hinge being greater than a torque caused by a weight of the holding structure and the radiation protection curtain with respect to the pivot axis.

One embodiment provides that the pivoting apparatus has a gantry-side region,
wherein the holding structure is pivotably mounted relative to the gantry-side region about the pivot axis via the pivoting apparatus,
wherein the gantry has a set of bolts,
wherein the gantry-side region has a set of recesses for receiving the set of bolts,
wherein the set of bolts is received in the set of recesses in such a way that the gantry-side region is secured in a form-fitting manner against displacement relative to the gantry in a plane which is substantially perpendicular to the system axis and/or to the bolts of the set of bolts.

The gantry-side region can, for example, be embodied as substantially rod-shaped and/or extend substantially along the pivot axis. The gantry-side region can, for example, be substantially made of aluminum.

One embodiment provides that the bolts of the set of bolts are aligned substantially parallel to the system axis. In particular, it can be provided that, for each bolt of the set of bolts, the axial direction of this bolt is substantially horizontal and/or substantially parallel to the system axis.

One embodiment provides that at least one bolt of the set of bolts has a groove,
wherein the gantry-side region has a securing element in at least one recess of the set of recesses,
wherein the securing element is inserted into the groove perpendicular to an axial direction of the at least one bolt in such a way that the gantry-side region is secured in a form-fitting manner against displacement relative to the gantry in the axial direction of the at least one bolt, in particular secured in a form-fitting manner against removal from the gantry in the axial direction of the at least one bolt.

One embodiment provides that the gantry-side region has a spring which is configured to press the securing element perpendicular to the axial direction of the at least one bolt against the groove.

One embodiment provides that the gantry-side region has an operating element which is arranged on a surface of the gantry-side region and is connected to the securing element in such a way that pressure on the operating element can cause the securing element to be pressed out of the groove, in particular against the spring force of the spring. The pressure on the operating element can, for example, be applied manually and/or by an operator's finger.

The holding structure can in particular be embodied as dimensionally stable. The pivot axis can, for example, be substantially perpendicular to the system axis, in particular perpendicular to the system axis. The pivot axis can, for example, be substantially parallel to the system axis, in particular parallel to the system axis. The pivot axis can, for example, be skewed with respect to the system axis and, herein, in particular be neither substantially perpendicular nor substantially parallel to the system axis.

The opening can, for example, be tunnel-shaped and/or extend along the system axis. The system axis can, for example, pass through the opening, in particular through a central region of the opening. The system axis can, for example, pass through an isocenter of the computed tomography device. In particular, it can be provided that the vertical position of the first edge of the holding structure is higher than a vertical position of the system axis both in the first position of the holding structure and in the second position of the holding structure.

The pivoting apparatus can in particular be a pivot bearing and/or embodied in the form of a folding mechanism. The radiation protection apparatus is designed such that, when the holding structure is in the first position relative to the gantry, a patient can be positioned relative to the gantry in such a way that the patient's head is in the opening, and such that, when the holding structure is in the second position relative to the gantry, the holding structure does not come into contact with the patient.

Such a radiation protection apparatus is easily accessible from both edge areas to the left and right of the patient bed where the operating personnel are located for the examination and/or to prepare for the examination, in particular both for lowering the holding structure from the first position and for raising the holding structure from the second position.

Furthermore, it can be provided that the holding structure is pivotably mounted relative to the gantry via the pivoting apparatus about the pivot axis in such a way that a second pivoting motion of the holding structure about the pivot axis enables the holding structure to be raised relative to the gantry from the second position to the first position.

One embodiment provides that the gantry has an inner region and paneling for separating the inner region from an environment, wherein the holding structure abuts a region of the paneling when the holding structure is in the first position of the holding structure, wherein the holding structure extends away from the region of the paneling when the holding structure is in the second position of the holding structure.

One embodiment provides that the holding structure extends flat in a cover plane and that the cover plane is substantially parallel to the pivot axis. For example, an angle between the cover plane and the system axis can be greater than 30 degrees and/or less than 60 degrees, in particular approximately 45 degrees, when the holding structure is in the second position of the holding structure. Another embodiment provides that the holding structure extends flat in a cover plane and that the cover plane and the pivot axis intersect at an angle that is significantly different from zero, for example approximately 45 degrees.

The pivot axis can, for example, be located in a region of a second edge of the holding structure. The first edge of the holding structure and the second edge of the holding structure can in particular be arranged opposite one another with respect to a central region of the holding structure. The distance between the first edge of the holding structure and the pivot axis can, for example, be greater than 10 cm, in particular greater than 20 cm, in particular greater than 30 cm. The distance between the first edge of the holding structure and a second edge of the holding structure can, for example, be greater than 10 cm, in particular greater than 20 cm, in particular greater than 30 cm.

Furthermore, it can be provided that the holding structure can be lowered further beyond the second position, for example in such a way that the holding structure can assume a position relative to the gantry in which the cover plane is substantially perpendicular, in particular perpendicular, to the system axis and/or in which the holding structure abuts an edge of the opening. This can, for example, be useful for a calibration measurement in which an examination object in the form of a phantom can be arranged in the opening without parts connected to the examination object protruding from the opening.

One embodiment provides that the system axis is substantially horizontal, in particular horizontal, and that the pivot axis is substantially horizontal, in particular horizontal. One embodiment provides that the pivot axis is located above the opening.

In particular, it can be provided that the vertical position of the first edge of the holding structure in the first position of the holding structure is higher than a vertical position of the pivot axis and/or that the vertical position of the first edge of the holding structure in the second position of the holding structure is lower than a vertical position of the pivot axis.

The radiation protection curtain enables, for example, gaps between the paneling of the gantry on the one hand and the patient and/or the patient bed on the other to be covered. The radiation protection curtain can in particular be made of a flexible sheet-like radiation protection material and/or be substantially impermeable to scatter radiation from the X-rays used in the computed tomography device for examining the examination object. The flexible sheet-like radiation protection material can, for example, be lead-free or contain lead. The radiation protection curtain can, for example, have a region transparent to visible light, for example in the form of a lead glass window.

One embodiment provides that a first lateral portion of the radiation protection curtain extends from the gantry to a first edge of the holding structure, in particular extends substantially parallel to the system axis, and that a second lateral portion of the radiation protection curtain extends from the gantry to the first edge of the holding structure, in particular extends substantially parallel to the system axis.

Furthermore, it can be provided that a front portion of the radiation protection curtain extends from the first lateral portion of the radiation protection curtain to the second lateral portion of the radiation protection curtain, in particular extends substantially perpendicular to the system axis. Furthermore, it can be provided that the system axis is located between the first lateral portion of the radiation protection curtain and the second lateral portion of the radiation protection curtain when the holding structure is in the second position of the holding structure.

Lowering and raising the holding structure causes the radiation protection curtain to be moved substantially vertically relative to the patient. This reduces the risk of cables and/or tubes connected to the patient being entrained and damaged by the radiation protection curtain. By being attached to the holding structure, the radiation protection curtain can be positioned relative to the patient in such a way that as little weight as possible of the radiation protection curtain is borne by the patient and that the weight of the radiation protection curtain borne by the patient is distributed as evenly as possible over the affected regions of the patient.

One embodiment provides that the gantry has a first gantry part, a second gantry part and a third gantry part, wherein the first gantry part has a rotatably mounted rotor with a projection data acquisition system, wherein the third gantry part has at least one section of the opening, wherein the holding structure is connected to the third gantry part via the pivoting apparatus and is pivotably mounted relative to the third gantry part about the pivot axis. The projection data acquisition system can, for example, have an X-ray source and an X-ray detector that interacts with the X-ray source.

Herein, the first gantry part can be movably mounted relative to the second gantry part and relative to the third gantry part in such a way that a translational motion of the first gantry part relative to the second gantry part and relative to the third gantry part can be executed while, at the same time, the second gantry part and the third gantry part are stationary relative to the examination object and the radiation protection apparatus is stationary relative to the examination object and relative to the at least one section of the opening when the examination object is located in the opening.

The examination object can, for example, be a body part of a patient, in particular a head of a patient. The computed tomography device can in particular be embodied as a head computed tomography device and/or as a mobile computed tomography device.

The patient can, for example, be a human being, in particular a baby, or an animal. A longitudinal axis of the patient can, for example, be parallel to the system axis, in particular identical or skewed, or intersect the system axis.

The examination object can, for example, be an object, in particular a phantom for calibrating the computed tomography device.

One embodiment provides that the first gantry part has a pivot bearing and a supporting structure, wherein the rotor is connected to the supporting structure via the pivot bearing and is rotatably mounted relative to the supporting structure about the system axis. The second gantry part can, for example, have a chassis and/or a head tray.

The computed tomography device can, for example, further have a lighting system configured to illuminate the opening at least when the holding structure is in the second position of the holding structure.

The lighting system can, for example, have a holding- structure-side light source attached to the holding structure in such a way that the light source illuminates the opening when the holding structure is in the second position relative to the gantry. The lighting system can, for example, have a rear light source attached to a radiation protection body that closes off the rear side of the opening.

The lighting system can be used to provide sufficient illumination of the opening even when the rear side of the opening is closed off by a radiation protection body that is opaque to visible light.

The computed tomography device can, for example, further have a camera system configured to optically capture the examination object at least when the examination object is located in the opening and when the holding structure is located in the second position of the holding structure.

The use of the camera system within the radiation protection region, for example within the opening, enables a patient to be observed even when visible light cannot penetrate outward from the opening or when a patient cannot be observed through a region of the holding structure that is transparent to visible light. The computed tomography device can in particular have a display. An image of the examination object generated by the camera system can, for example, be shown on the display.

The camera system can, for example, have a camera attached to the gantry and/or a camera attached to the holding structure and/or a camera attached to a radiation protection body which closes off the rear side of the opening. The camera attached to the gantry can in particular be attached to a portion of the gantry paneling that delimits the opening. Furthermore, it can be provided that the camera system has at least one camera light source, thereby forming the lighting system. The camera light source can, for example, have a ring of light-emitting diodes surrounding a lens of the camera in a ring shape.

The computed tomography device can, for example, further have an acoustic system configured to transmit an acoustic signal to the examination object and/or to receive an acoustic signal emanating from the examination object at least when the examination object is in the opening and when the holding structure is in the second position of the holding structure.

The acoustic system can, for example, have a microphone and/or a loudspeaker. The microphone can, for example, be attached to the gantry or to the holding structure or to a radiation protection body that closes off the rear side of the opening. The loudspeaker can, for example, be attached to the gantry or to the holding structure or to a radiation protection body that closes off the rear side of the opening.

The computed tomography device can, for example, further have an optical system configured to transmit an optical signal to the examination object at least when the examination object is in the opening and when the holding structure is in the second position of the holding structure.

The optical system can, for example, have a display for displaying optical signals in the form of text and/or images and/or a projector for projecting optical signals in the form of light beams, in particular laser beams. The display can, for example, be attached to the gantry or to the holding structure or to a radiation protection body that closes off the rear side of the opening. The projector can, for example, be attached to the gantry or to the holding structure or to a radiation protection body that closes off the rear side of the opening.

Furthermore, the computed tomography device can have a radiation protection body that can be detachably connected to the gantry in such a way that it covers a rear side of the opening. The rear side of the opening can in particular be located on a rear side of the gantry, wherein a front side of the gantry faces the radiation protection apparatus. In particular, it can be provided that the first gantry part has the rear side of the opening and that the third gantry part has the front side of the opening. The radiation protection body can, for example, have a power supply interface and/or a data transmission interface for at least one component of the lighting system, the camera system, the acoustic system and/or the optical system.

Within the scope of present invention, features described with respect to different embodiments of the present invention and/or different categories of claims (method, use, apparatus, system, arrangement, etc.) can be combined to form further embodiments of the present invention. For example, a claim relating to an apparatus can also be developed with features described or claimed in connection with a method and vice versa. Herein, functional features of a method can be executed by correspondingly embodied physical components. The use of the indefinite articles “a” or “an” does not exclude the possibility that the feature concerned may also be present more than once.

### BRIEF DESCRIPTION OF THE DRAWINGS

The following describes features of the present invention with reference to examples and with reference to the attached figures. The representation in the figures is schematic, greatly simplified and not necessarily true to scale.

FIG. 1 shows a computed tomography device with a gantry and a radiation protection apparatus for covering an opening.

FIGS. 2 to 9 show different views of parts of the radiation protection apparatus and/or the gantry.

FIG. 10 shows another view of a computed tomography device with a gantry and a radiation protection apparatus for covering an opening.

FIG. 11 shows another view of a computed tomography device with a gantry and a radiation protection apparatus for covering an opening.

### DETAILED DESCRIPTION

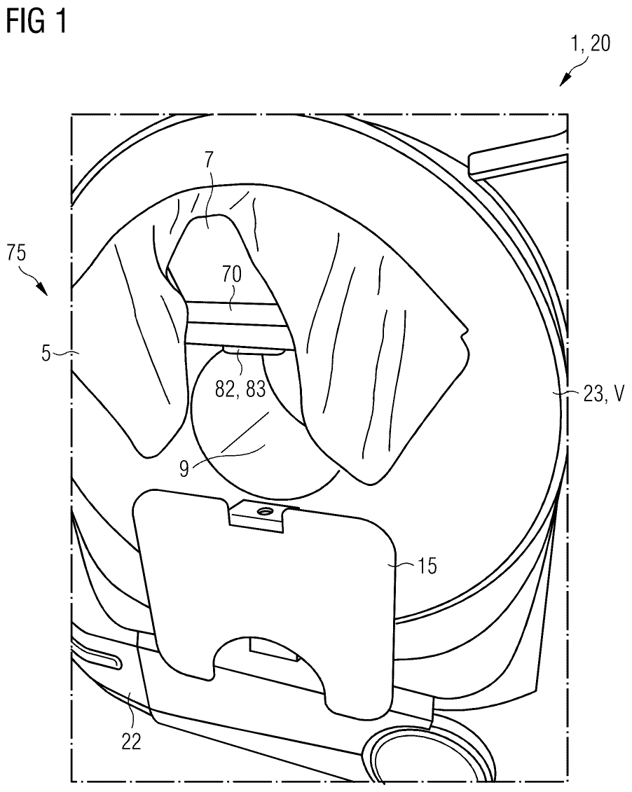

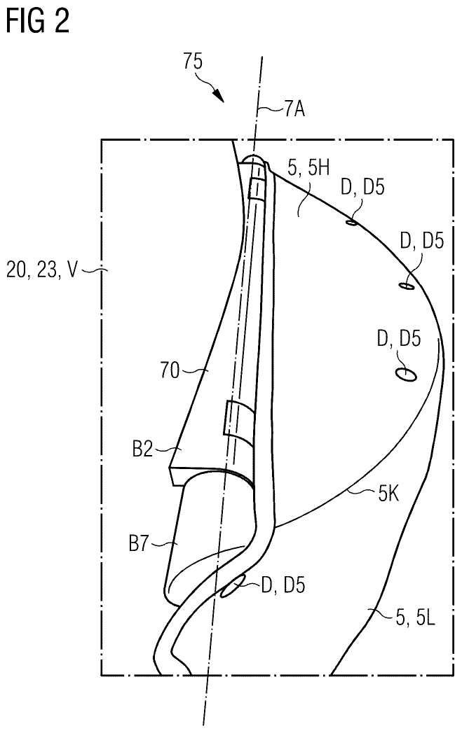

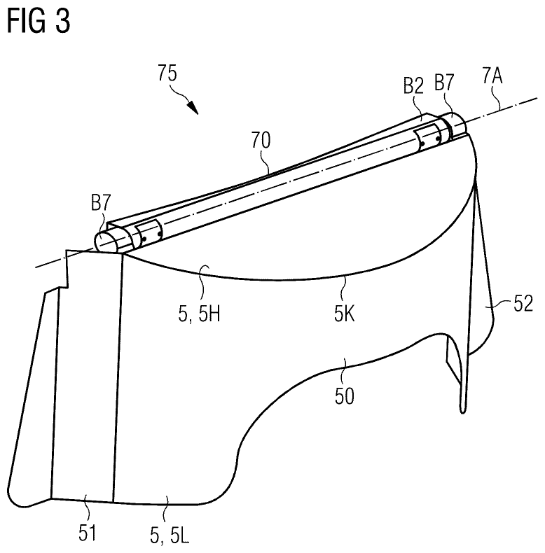

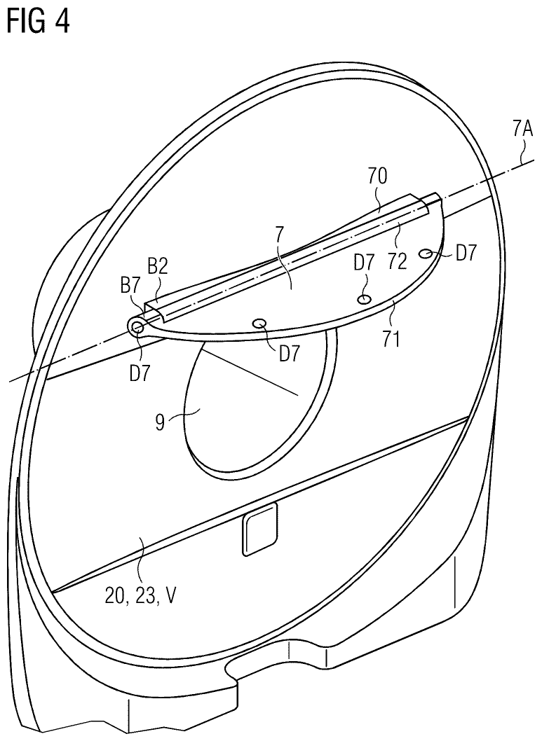

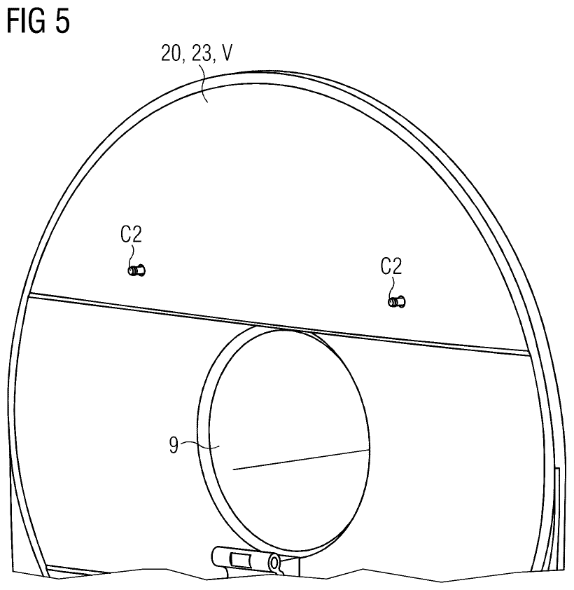

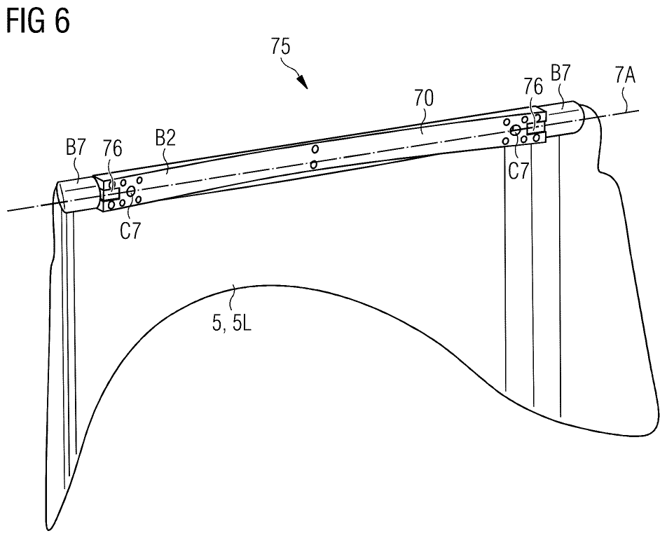

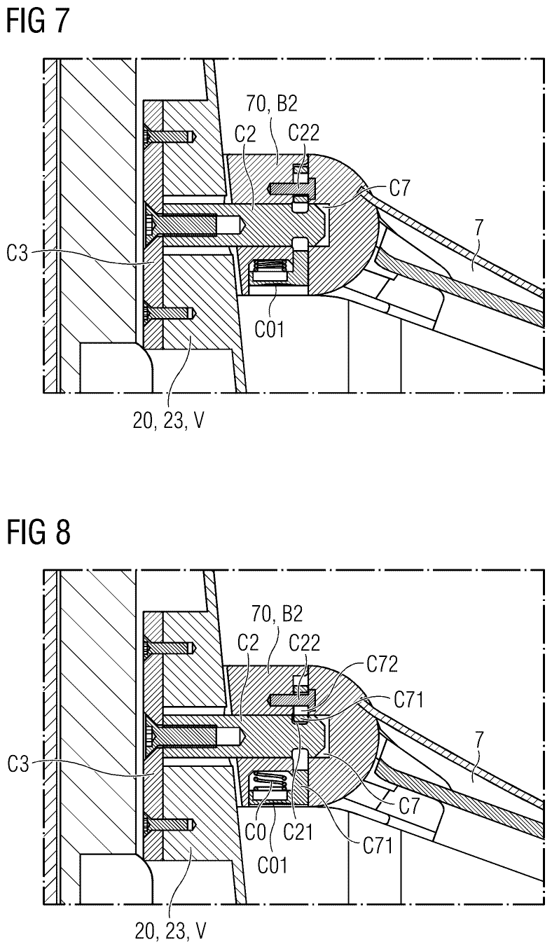

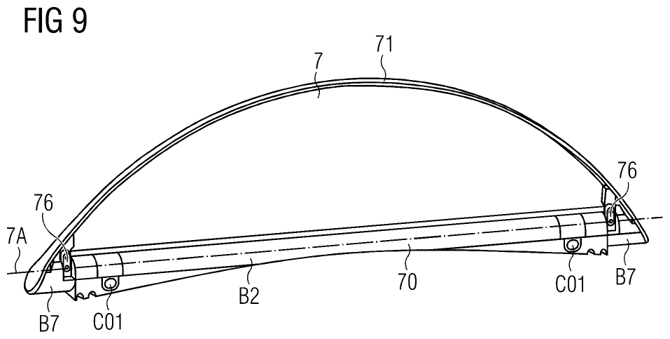

FIG. 1 shows the computed tomography device 1 with the gantry 20 and the radiation protection apparatus 75 for covering the opening 9, wherein the holding structure 7 is in the first position. FIGS. 2 to 9 show different views of parts of the radiation protection apparatus 75 and/or the gantry 20.

The protruding holding structure 7 in the second position creates corresponding space below the radiation protection curtain 5. Hence, there is more space under the radiation protection curtain 5 for, for example, a tube, hoses and similar accessories for the medical imaging examination.

The radiation protection apparatus 75 has the radiation protection curtain 5, wherein the radiation protection curtain 5 is attached to the holding structure 7 and hangs down from the holding structure 7. A first lateral portion 51 of the radiation protection curtain 5 extends from the gantry 20 to a first edge 71 of the holding structure 7. A second lateral portion 52 of the radiation protection curtain 5 extends from the gantry 20 to the first edge 71 of the holding structure 7. A front portion 50 of the radiation protection curtain 5 extends from the first lateral portion 51 of the radiation protection curtain 5 to the second lateral portion 52 of the radiation protection curtain 5. The system axis SA is located between the first lateral portion 51 of the radiation protection curtain 5 and the second lateral portion 52 of the radiation protection curtain 5 when the holding structure 7 is in the second position of the holding structure 7.

The pivot axis 7A is located in a region of a second edge 72 of the holding structure 7. The first edge 71 and the second edge 72 are arranged opposite one another with respect to a central region of the holding structure 7.

The computed tomography device 1 according to the example shown has a gantry 20 with an opening 9 and a radiation protection apparatus 75 for covering the opening 9,
wherein the radiation protection apparatus 75 has a holding structure 7, a radiation protection curtain 5 and a pivoting apparatus 70,
wherein an examination object 14 can be inserted into the opening 9 along a system axis SA of the gantry 20 when the holding structure 7 is in a first position relative to the gantry 20,
wherein the holding structure 7 is pivotably mounted relative to the gantry 20 about a pivot axis 7A via the pivoting apparatus 70 in such a way that the holding structure 7 can be lowered relative to the gantry 20 from the first position to a second position by a first pivoting motion of the holding structure 7 about the pivot axis 7A,
wherein the radiation protection curtain 5 is arranged on the holding structure 7, in particular is attached to the holding structure 7,
wherein a lower region 5L of the radiation protection curtain 5 hangs down from the holding structure 7,
wherein an upper region 5H of the radiation protection curtain 5 rests on the holding structure 7.

The example shown provides that the upper region 5H of the radiation protection curtain 5 rests on the holding structure 7 in such a way that the upper region 5H of the radiation protection curtain 5 shields a region 8 of an environment U of the gantry 20 from scatter radiation from X-rays 4 which emanate from the opening 9 and are directed toward the region 8 of the environment U of the gantry 20 and penetrate the holding structure 7, in particular when the holding structure 7 is in the second position of the holding structure 7.

The example shown provides that the lower region 5L of the radiation protection curtain 5 and the upper region 5H of the radiation protection curtain 5 adjoin one another along an edge 5K of the radiation protection curtain 5,
wherein the radiation protection curtain 5 is arranged on the holding structure 7 in such a way that the edge 5K of the radiation protection curtain 5 extends along a first edge 71 of the holding structure 7.

In particular, it can be provided that a vertical position of the first edge 71 of the holding structure 7 is lower in the second position of the holding structure 7 than in the first position of the holding structure 7. The first edge 71 of the holding structure 7 can in particular be located on a side of the holding structure 7 facing away from the pivot axis 7A. The pivot axis 7A can, for example, be located in a region of a second edge 72 of the holding structure 7. In particular, it can be provided that the upper region 5H of the radiation protection curtain 5 extends from the first edge 71 of the holding structure 7 to the pivot axis 7A and/or to the second edge 72 of the holding structure 7.

The example shown provides that the lower region 5L of the radiation protection curtain 5 and the upper region 5H of the radiation protection curtain 5 are sewn together at the edge 5K of the radiation protection curtain 5. The example shown provides that the holding structure 7 extends flat, wherein the upper region 5H of the radiation protection curtain 5 rests flat on the holding structure 7. The example shown provides that the holding structure 7 is made of plastic, in particular carbon fiber-reinforced plastic.

The example shown provides that the radiation protection curtain 5 is attached to the holding structure 7 via a set of pushbutton connections D. The example shown provides that the radiation protection curtain 5 has a set of curtain-side connecting elements D5,
wherein the holding structure 7 has a set of holding-structure-side connecting elements D7, wherein the set of curtain-side connecting elements D5 and the set of holding-structure-side connecting elements D7 together form the set of pushbutton connections D.

The example shown provides that the set of holding-structure-side connecting elements D7 has a first row of holding-structure-side connecting elements D7, wherein the first row of holding-structure-side connecting elements D7 is arranged following a course of the first edge 71 of the holding structure 7, wherein the set of curtain-side connecting elements D5 has a first row of curtain-side connecting elements D5, wherein the first row of curtain-side connecting elements D5 is arranged following a course of the edge 5K of the radiation protection curtain 5 and corresponding to the first row of holding-structure-side connecting elements D7.

The example shown provides that the pivoting apparatus 70 has at least one friction hinge 76,
wherein the holding structure 7 is pivotably mounted relative to the gantry 20 about the pivot axis 7A via the at least one friction hinge 76,
wherein the at least one friction hinge 76 is configured to hold the holding structure 7 in the second position relative to the gantry 20, in particular by a breakaway torque of the friction hinge 76 being greater than a torque caused by a weight of the holding structure 7 and the radiation protection curtain 5 with respect to the pivot axis 7A.

The example shown provides that the pivoting apparatus 70 has a gantry-side region B2,
wherein the holding structure 7 is pivotably mounted relative to the gantry-side region B2 about the pivot axis 7A via the pivoting apparatus 70,
wherein the gantry 20 has a set of bolts C2,
wherein the gantry-side region B2 has a set of recesses C7 for receiving the set of bolts C2,
wherein the set of bolts C2 is received in the set of recesses C7 in such a way that the gantry-side region B2 is secured in a form-fitting manner against displacement relative to the gantry 20 in a plane which is substantially perpendicular to the system axis SA and/or to the bolts of the set of bolts C2.

The gantry-side region B2 can, for example, be embodied as substantially rod-shaped and/or extend substantially along the pivot axis 7A. The gantry-side region B2 can, for example, be substantially made of aluminum.

The example shown provides that the bolts of the set of bolts C2 are aligned substantially parallel to the system axis SA.

The example shown provides that at least one bolt of the set of bolts C2 has a groove C21,
wherein the gantry-side region B2 has a securing element C71 in at least one recess of the set of recesses C7, wherein the securing element C71 is inserted into the groove C21 perpendicular to an axial direction of the at least one bolt in such a way that the gantry-side region B2 is secured in a form-fitting manner against displacement relative to the gantry 20 in the axial direction of the at least one bolt, in particular secured in a form-fitting manner against removal from the gantry 20 in the axial direction of the at least one bolt.

The example shown provides that the gantry-side region B2 has a spring C0 which is configured to press the securing element C71 perpendicular to the axial direction of the at least one bolt against the groove C21.

The example shown provides that the gantry-side region B2 has an operating element C01 which is arranged on a surface of the gantry-side region B2 and is connected to the securing element C71 in such a way that pressure on the operating element C01 can cause the securing element C71 to be pressed out of the groove C21, in particular against the spring force of the spring.

The metal plate C3 is screwed to the paneling V from the inside. Each bolt of the set of bolts C2 is screwed to a corresponding metal plate C3. The securing element C71 comprises a plate-shaped region with corresponding holes and engages in the groove C21. The spring CO holds the securing element C71 in the groove C21. Hence, the gantry-side region B2 is fixed relative to the gantry 20. For cleaning purposes and/or for using the gantry 20 without the radiation protection apparatus 75, for example in an ambulance, the radiation protection apparatus 75 can thus be easily detached from the gantry 20.

The plate-shaped region of the securing element C71 extends flat in a plane perpendicular to the axial direction of the bolt C2 and has a circular hole through which the bolt C2 extends. The plate-shaped region of the securing element C71 further has the elongated hole C72 which extends perpendicular to the axial direction of the bolt C2. The screw C22 extends through the elongated hole C72 and, together with the elongated hole C72, forms a guide for the securing element C71.

The gantry-side region B2 is connected to the gantry-side portion of the at least one friction hinge 76. The holding-structure-side portion of the at least one friction hinge 76 can, for example, be screwed to a threaded plate which is laminated into the holding structure 7, in particular laminated into a corresponding end piece B7 of the holding structure 7. A holding-structure-side connecting element of the set of holding-structure-side connecting elements D7 is attached to each of the two end pieces B7 of the holding structure 7 in each case.

The at least one friction hinge 76 provides sufficient holding force so that the holding structure 7 can be held relative to the gantry in various positions, in particular in each position that is passed through during the first pivoting motion from the first position to the second position. Hence, the holding structure 7 can be held in different angular positions relative to the gantry 20. In particular, it can be provided that the at least one friction hinge 76 can hold the holding structure 7 in each position between 90 degrees upward relative to the system axis SA and 90 degrees downward relative to the system axis SA.

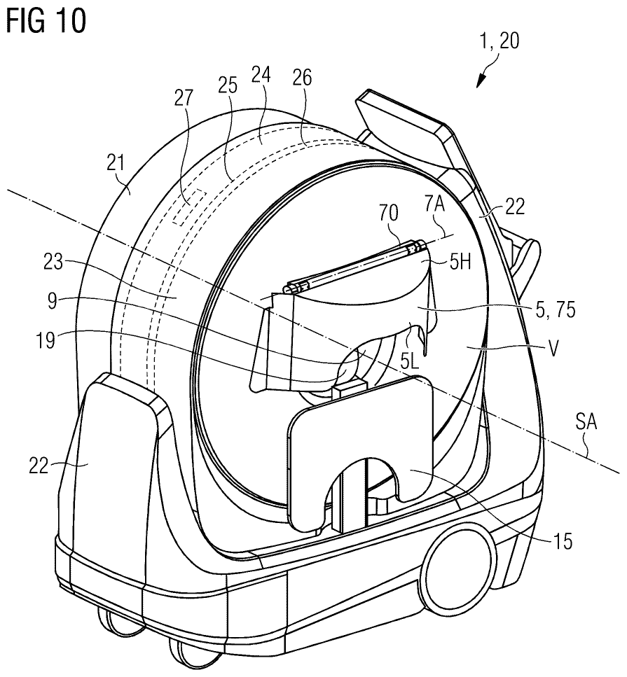

FIG. 10 shows a computed tomography device with a gantry and a radiation protection apparatus for covering an opening in a further view, wherein the holding structure 7 is located in the second position. The examination object 14 is the head of the patient 13. The computed tomography device 1 is a mobile head computed tomography device. The computed tomography device 1 further has a head tray 19, in which the head of the patient 13 can be accommodated, and an upper body support plate 15, on which the upper body of the patient 13 can be supported.

The gantry 20 has a first gantry part 21, a second gantry part 22 and a third gantry part 23, wherein the first gantry part 21 has a rotatably mounted rotor 24 with a projection data acquisition system 27, wherein the third gantry part 23 has at least one section of the opening 9, wherein the holding structure 7 is connected to the third gantry part 23 via the pivoting apparatus 70 and is pivotably mounted relative to the third gantry part 23 about the pivot axis 7A.

The first gantry part 21 is movably mounted relative to the second gantry part 22 and relative to the third gantry part 23 in such a way that a translational motion of the first gantry part 21 relative to the second gantry part 22 and the third gantry part 23 can be executed while, at the same time, the second gantry part 22 and the third gantry part 23 are stationary relative to the examination object 14 and the radiation protection apparatus 75 is stationary relative to the examination object 14 and relative to the at least one section of the opening 9 when the examination object 14 is located in the opening 9.

The first gantry part 21 has a pivot bearing 25 and a supporting structure 26, wherein the rotor 24 is connected to the supporting structure 26 via the pivot bearing 25 and rotatably mounted relative to the supporting structure 26 about the system axis SA. The projection data acquisition system 27 has the X-ray source 4A for generating the X-rays 4 and the X-ray detector 4B for detecting the X-rays 4.

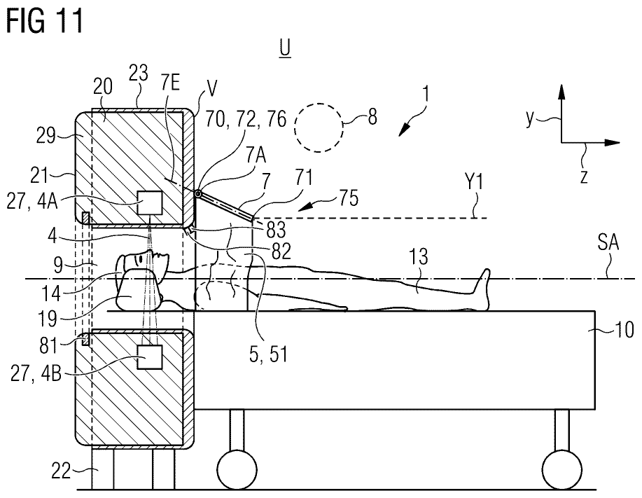

FIG. 11 shows a computed tomography device with a gantry and a radiation protection apparatus for covering an opening in a further view, wherein the holding structure 7 is located in the second position.

The computed tomography device 1 has the patient bed 10 for supporting the patient 13. The gantry 20 has an inner region 29 and paneling V for separating the inner region 29 from an environment U, wherein the holding structure 7 abuts a region of the paneling V when the holding structure 7 is in the first position of the holding structure 7, wherein the holding structure 7 projects away from the region of the paneling V when the holding structure 7 is in the second position of the holding structure 7.

The holding structure 7 extends flat in a cover plane 7E, wherein the cover plane 7E is substantially parallel to the pivot axis 7A. The system axis SA is horizontal and parallel to the horizontal direction z. The pivot axis 7A is horizontal and perpendicular to the horizontal direction z. The pivot axis 7A is located above the opening 9.

The vertical position Y1 of the first edge 71 of the holding structure 7 is lower in the second position of the holding structure 7 than in the first position of the holding structure 7. With respect to the vertical direction y, there is a space between the patient 13 and the holding structure 7. This reduces the risk of the patient 13 colliding with the holding structure 7.

The computed tomography device 1 further has a lighting system 81 configured to illuminate the opening 9 at least when the holding structure 7 is in the second position of the holding structure 7.

The computed tomography device 1 further has a camera system 82 configured to optically capture the examination object 14 at least when the examination object 14 is located in the opening 9 and when the holding structure 7 is located in the second position of the holding structure 7. The camera system 82 has a camera attached to the holding structure 7. The computed tomography device 1 has a screen. An image of the examination object 14 generated by the camera system 82 can, for example, be displayed on a screen.

The computed tomography device 1 further has an acoustic system 83 configured to transmit an acoustic signal to the examination object 14 and/or to receive an acoustic signal emanating from the examination object 14 at least when the examination object 14 is in the opening 9 and when the holding structure 7 is in the second position of the holding structure 7. The acoustic system 83 has a microphone and a loudspeaker which are attached to the holding structure 7.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term

computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

What is claimed is:

1. A computed tomography device, comprising:
a gantry with an opening; and
a radiation protection apparatus configured to cover the opening, wherein
the radiation protection apparatus has a holding structure, a radiation protection curtain and a pivoting apparatus,
the gantry is configured to receive an examination object inserted into the opening along a system axis of the gantry when the holding structure is in a first position relative to the gantry,
the holding structure is pivotably mounted relative to the gantry via the pivoting apparatus about a pivot axis such that the holding structure is configured to be lowered, relative to the gantry, from the first position to a second position, by a first pivoting motion of the holding structure about the pivot axis,
the radiation protection curtain is arranged on the holding structure,
a lower region of the radiation protection curtain hangs down from the holding structure, and
an upper region of the radiation protection curtain rests on the holding structure.

2. The computed tomography device as claimed in claim 1, wherein the upper region of the radiation protection curtain rests on the holding structure such that the upper region of the radiation protection curtain shields a region of an environment of the gantry from scatter radiation from X-rays which, emanating from the opening, are directed toward the region of the environment of the gantry and penetrate the holding structure.

3. The computed tomography device as claimed in claim 1, wherein
the lower region of the radiation protection curtain and the upper region of the radiation protection curtain adjoin one another along an edge of the radiation protection curtain, and
the radiation protection curtain is arranged on the holding structure such that the edge of the radiation protection curtain extends along a first edge of the holding structure.

4. The computed tomography device as claimed in claim 3, wherein the lower region of the radiation protection curtain and the upper region of the radiation protection curtain are sewn together at the edge of the radiation protection curtain.

5. The computed tomography device as claimed in claim 1, wherein
the holding structure extends flat, and
the upper region of the radiation protection curtain rests flat on the holding structure.

6. The computed tomography device as claimed in claim 1, wherein the holding structure is made of plastic.

7. The computed tomography device as claimed in claim 1, wherein the radiation protection curtain is attached to the holding structure via a set of pushbutton connections.

8. The computed tomography device as claimed in claim 7, wherein
the radiation protection curtain has a set of curtain-side connecting elements,
the holding structure has a set of holding-structure-side connecting elements, and
the set of curtain-side connecting elements and the set of holding-structure-side connecting elements together form the set of pushbutton connections.

9. The computed tomography device as claimed in claim 8, wherein
the set of holding-structure-side connecting elements has a first row of holding-structure-side connecting elements,
the first row of holding-structure-side connecting elements is arranged to follow a first edge of the holding structure,
the set of curtain-side connecting elements has a first row of curtain-side connecting elements, and
the first row of curtain-side connecting elements is arranged to follow an edge of the radiation protection curtain and corresponds to the first row of holding-structure-side connecting elements.

10. The computed tomography device as claimed in claim 1, wherein
the pivoting apparatus has at least one friction hinge,
the holding structure is pivotably mounted relative to the gantry about the pivot axis via the at least one friction hinge, and
the at least one friction hinge is configured to hold the holding structure in the second position relative to the gantry.

11. The computed tomography device as claimed in claim 1, wherein
the pivoting apparatus has a gantry-side region,
the holding structure is pivotably mounted relative to the gantry-side region about the pivot axis via the pivoting apparatus,
the gantry has a set of bolts,
the gantry-side region has a set of recesses configured to receive the set of bolts, and
the set of bolts is configured to secure the gantry-side region, in a form-fitting manner, against displacement relative to the gantry in a plane that is substantially perpendicular to at least one of the system axis or to bolts of the set of bolts.

12. The computed tomography device as claimed in claim 11, wherein the bolts of the set of bolts are aligned substantially parallel to the system axis.

13. The computed tomography device as claimed in claim 11, wherein
at least one bolt of the set of bolts has a groove,
the gantry-side region has a securing element in at least one recess of the set of recesses, and
the securing element is inserted into the groove perpendicular to an axial direction of the at least one bolt such that the gantry-side region is secured, in a form-fitting manner, against displacement relative to the gantry in the axial direction of the at least one bolt.

14. The computed tomography device as claimed in claim 13, wherein the gantry-side region has a spring that is configured to press the securing element against the groove, perpendicular to the axial direction of the at least one bolt.

15. The computed tomography device as claimed in claim 13, wherein the gantry-side region has an operating element arranged on a surface of the gantry-side region, the operating element being connected to the securing element such that pressure on the operating element causes the securing element to be pressed out of the groove.

16. The computed tomography device as claimed in claim 2, wherein
the lower region of the radiation protection curtain and the upper region of the radiation protection curtain adjoin one another along an edge of the radiation protection curtain, and
the radiation protection curtain is arranged on the holding structure such that the edge of the radiation protection curtain extends along a first edge of the holding structure.

17. The computed tomography device as claimed in claim 16, wherein the lower region of the radiation protection curtain and the upper region of the radiation protection curtain are sewn together at the edge of the radiation protection curtain.

18. The computed tomography device as claimed in claim 2, wherein
the pivoting apparatus has at least one friction hinge,
the holding structure is pivotably mounted relative to the gantry about the pivot axis via the at least one friction hinge, and
the at least one friction hinge is configured to hold the holding structure in the second position relative to the gantry.

19. The computed tomography device as claimed in claim 2, wherein
the pivoting apparatus has a gantry-side region,
the holding structure is pivotably mounted relative to the gantry-side region about the pivot axis via the pivoting apparatus,
the gantry has a set of bolts,
the gantry-side region has a set of recesses configured to receive the set of bolts, and
the set of bolts is configured to secure the gantry-side region, in a form-fitting manner, against displacement relative to the gantry in a plane that is substantially perpendicular to at least one of the system axis or to bolts of the set of bolts.

20. The computed tomography device as claimed in claim 12, wherein
at least one bolt of the set of bolts has a groove,
the gantry-side region has a securing element in at least one recess of the set of recesses, and
the securing element is inserted into the groove perpendicular to an axial direction of the at least one bolt such that the gantry-side region is secured, in a form-fitting manner, against displacement relative to the gantry in the axial direction of the at least one bolt.
